(19) United States
(12) Patent Application Publication
(10) Pub. No.: US 2016/0186209 A1
(43) Pub. Date: Jun. 30, 2016

(54) RECOMBINANT CELLS AND METHODS OF USING SUCH CELLS TO IDENTIFY CIRCADIAN RHYTHM MODULATORS

(71) Applicant: University of Memphis Research Foundation, Memphis, TN (US)

(72) Inventors: Andrew C. Liu, Memphis, TN (US); Chidambaram Ramanathan, Memphis, TN (US); John B. Hogenesch, Philadelphia, PA (US)

(21) Appl. No.: 14/391,944

(22) PCT Filed: Apr. 25, 2013
(86) PCT No.: PCT/US2013/038224

§ 371 Date: Oct. 10, 2014

Related U.S. Application Data
(60) Provisional application No. 61/638,674, filed on Apr. 26, 2012.

(51) Int. Cl. C12N 15/86; C12N 15/113; G01N 33/50

(52) U.S. Cl. CPC C12N 15/86; G01N 33/5067; G01N 33/5044; C12N 15/113; C12N 2740/15043; C12N 2320/12; C12N 2310/14

(57) ABSTRACT

The invention provides recombinant cells comprising detectable reporters useful in identifying agents, genes, and other modulators of circadian period length and amplitude. Such modulators are useful for resetting the circadian clock in a variety of contexts (e.g., jet lag, shift work). Such cells are also useful in selecting an administration regimen for a therapeutic agent, where the agent's efficacy and/or adverse side effects show circadian effects.

### CROSS-REFERENCE TO RELATED APPLICATIONS

The present application claims the benefit of and priority to U.S. Patent Application No. 61/638,674, filed Apr. 26, 2012, which is incorporated herein by reference in its entirety.

### STATEMENT OF RIGHTS TO INVENTIONS MADE UNDER FEDERALLY SPONSORED RESEARCH

This work was supported by the following grants from the National Institutes of Health, Grant Nos: NIH/NINDS 2R01NS054794-06 and NSF: IOS0920417. The government has certain rights in the invention.

### BACKGROUND OF THE INVENTION

In mammals, many aspects of behavior and physiology, such as the sleep-wake cycle, body temperature, blood pressure, and liver metabolism, are regulated by endogenous circadian clocks. The circadian time-keeping system is a hierarchical, multioscillator network with the central clock in the suprachiasmatic nucleus synchronizing and coordinating peripheral clocks. This is accomplished through neuronal connections, as well as humoral factors. Virtually all cells in the body are circadian oscillators. Nevertheless, cellular oscillators in different tissues are physiologically distinctive. The only high amplitude mammalian cellular clock model has been established in fibroblasts. This cellular model fails to provide an adequate platform for investigating clock function and identifying modulators of circadian rhythms in other cell types that are known to have rhythms.

### SUMMARY OF THE INVENTION

As described below, the present invention features recombinant cells comprising detectable reporters that facilitate high temporal resolution quantitative luminescence recording (including imaging) and methods of using such cells to identify modulators of circadian period length and amplitude.

In one aspect, the invention generally features a recombinant cell containing an expression vector, where the expression vector comprises a promoter selected from the group consisting of Period2 (Per2), Cry1, Cry1-Intron, and Bmal1, where the promoter is operationally linked to a detectable reporter that is expressed at high-amplitude and with a persistent rhythm.

In another aspect, the invention features a recombinant adipocyte or hepatocyte cell or progenitor thereof containing an expression vector, where the expression vector comprises Period2 (Per2), Cry1, Cry1-Intron, and Bmal1 promoter operationally linked to a detectable reporter (e.g., luciferase, GFP, YFP, RFP). In one embodiment, the cell is a 3T3-L1 pre-adipocyte or a MMH-D3 pre-hepatocyte. In another embodiment, the expression vector is a lentiviral vector In another embodiment, the reporter expression varies at least about two to four-fold (e.g., 2, 3, 4, 5, 6-fold) in trough to peak levels. In one embodiment, the reporter expression varies at least about three fold in trough to peak levels.

In another aspect, the invention features a method of identifying a circadian cycle modulator, the method involving contacting the cell of any previous aspect with an agent, and assaying reporter expression in the contacted cell relative to a corresponding control cell. In one embodiment, the agent is a small compound, inhibitory nucleic acid, or polypeptide.

In another aspect, the invention features a method of identifying a circadian cycle modulator, the method involving contacting the cell of any previous aspect with an shRNA against a gene of interest, and analyzing a circadian rhythm of the cell relative to a reference, thereby identifying a circadian cycle modulator. In one embodiment, the circadian rhythm of the cell is analyzed by detecting the amplitude, period length and phase of reporter expression. In another embodiment, the reference is the circadian rhythm of an untreated control cell. In another embodiment, the circadian rhythm is analyzed using luminescence recording, and/or real-time imaging. In another embodiment, the circadian cycle modulator is an inhibitory nucleic acid molecule, small compound, or polypeptide. In another embodiment, the inhibitory nucleic acid molecule is an shRNA.

The invention provides recombinant cells comprising detectable reporters useful in identifying agents, genes, and other modulators of circadian period length and amplitude. Such modulators are useful for resetting the circadian clock in a variety of contexts (e.g., jet lag, shift work). Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label or reporter” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The invention provides cells useful in identifying modulators of circadian rhythms, including genetic targets that are useful for the development of agents capable of altering a circadian rhythm. Such alteration can be at the cellular level or at the level of the organism. In addition, the methods of the invention provide a facile means to identify therapies that are safe for use in subjects. In addition, the compositions and methods of the invention provide a route for analyzing virtually any number of compounds for effects on circadian rhythms with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to a material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compositions or methods provided herein can be combined with one or more of any of the other compositions and methods provided herein.

### BRIEF DESCRIPTION OF THE DRAWINGS

FIGS. 1A-1C are graphs showing that fibroblasts, adipocytes, and hepatocytes display bioluminescence rhythms. FIG. 1A shows representative bioluminescence rhythms of reporter cells recorded in a LumiCycle luminometer on 35 mm dishes. Reporter cells were generated via lentiviral infection of either Per2-dLuc or Bmal1-dLuc luciferase reporter, and infected cell populations were recorded in a LumiCycle. Baseline-subtracted bioluminescence data of both reporter lines are plotted together to show the expected, approximately anti-phasic reporter expression for each cell type. FIG. 1B shows representative bioluminescence rhythms of homogenous clonal cell lines recorded in a Synergy microplate reader on 96-well plates. Baseline-subtracted bioluminescence data of selected clonal lines representing both reporter types are plotted together to show anti-phasic reporter expression for each cell type. High reproducibility is illustrated by showing data from 24 of the 96 wells for each reporter. FIG. 1C shows that mature adipocytes and hepatocytes are responsive to insulin treatment. Differentiated 3T3-L1 and MMH-D3 cells were treated with 0.1, 1, or 10 nM insulin for 5, 15, or 30 minutes, followed by cell lysis and Western blot analysis with ERK and pERK antibodies. Treatment with 0.1 nM insulin for 5 minutes was sufficient to activate ERK as reflected by its phosphorylation. Data are representative of two independent experiments.

FIGS. 2A and 2B provides a diagram of a lentiviral pLL3.7 Gateway vector and shows expression of the vector in infected cells. Only the region of integration in host cell's genome is shown (FIG. 2A). The shRNA expression cassette consists of sense target sequence, a loop and antisense sequence, and is driven by the mouse U6 promoter. EGFP expression is controlled by the CMV promoter. Typically, most infected cells are GFP positive as shown in (FIG. 2B).

FIG. 3A-3D show lentiviral shRNA-mediated knockdown of several known clock genes in 3T3 reporter cells. FIGS. 3A and 3C are Western blots showing shRNA-mediated knockdown of protein expression. Flag-tagged cDNA was co-transfected with the indicated shRNA in 3T3 cells, and protein expression was determined by Western blot using anti-Flag antibody. FIGS. 3B and 3D show shRNA-mediated knockdown effects on circadian phenotypes. 3T3 cells harboring a P(Bmal1)-dLuc reporter were infected with lentiviral shRNAs and recorded on Synergy in 96-well plates. NS, non-specific shRNA. Highlighted (red and green) for each gene are two shRNAs that down-regulated protein expression (FIGS. 3A and 3C) and produced circadian phenotypes (FIGS. 3B and 3D).

FIG. 4 is a graph showing results of a mammalian two-hybrid assay or Gal4 trap. Bait and prey constructs were generated for the indicated known clock components and their interactions were tested in 293T cells. pBIND: fusion constructs with Gal4 DNA-binding domain (DBD) (bait). pACT: fusion with VP16 trans-activation domain (TAD) (prey). Reciprocal interactions are tested.

FIG. 5 shows four circadian phases of gene expression. 3T3 cells were introduced with the indicated lentiviral reporters, each harboring a different promoter. Raw bioluminescence data are plotted together to show the different phases (activity peaks). Note that P(Cry1)-Intron reports a phase in between P(Cry1) and P(Bmal1).

FIG. 6A-6D are promoter sequences used in the lentiviral reporters. FIG. 6E shows the vector maps used in the Examples.

FIGS. 7A, 7B and 7C show that knockdowns of Bmal1, Clock, Cry1, Cry2, and Fbxl3 lead to cell type-ubiquitous circadian phenotypes. Bioluminescence expression patterns upon knockdown of Bmal1 or Clock (FIG. 7A), Cry1 or Cry2 (FIG. 7B), and Fbxl3 (FIG. 7C) in all three cell types. For clock phenotyping, both reporters were used for each cell line and phenotypes were independent of the reporter used. For the figure, we selected 3T3 and 3T3-L1 cell lines each expressing the Bmal1-dLuc reporter, and MMH-D3 expressing the Per2-dLuc reporter. Cells were infected with specific lentiviral shRNAs as indicated. Real-time bioluminescence expression was recorded by Synergy microplate reader as in FIG. 1. Out of the 5 shRNAs tested, two validated shRNAs (orange and green) are shown. NS, non-specific shRNA (black). While knockdown of Bmal1 or Clock resulted in low amplitude or arrhythmicity, Fbxl3 knockdown led to long periods or rapid damping. Cry1 knockdown caused short periods or rapid loss of rhythmicity, and Cry2 knockdown lengthened period and increased rhythm amplitude. Bioluminescence data are representative of six independent experiments for 3T3 cells and three independent experiments for 3T3-L1 and MMH-D3 cells. Knockdown of endogenous mRNA expression was determined by qPCR (insert). qPCR data are representative of two samples from one experiment.

FIGS. 8A-8C show the results of shRNA-mediated knockdowns of Per1, Per2 and Per3 lead to cell type-specific circadian phenotypes. Bioluminescence expression patterns upon knockdown of Per1 (FIG. 8A), Per2 (FIG. 8B), and Per3 (FIG. 8C) in all three cell types. Whereas Per3 knockdown led to short periods in all three cell types, Per1 and Per2 knockdown caused different clock phenotypes depending on cell type.

FIG. 9 shows the results of shRNA-mediated single and composite knockdown effects of Per1, Per2 and Per3 in MMH-D3 cells. Bioluminescence expression patterns on Lumicycle upon knockdown of Per1, Per2, Per3 (single KD) Per1/Per2, Per1/Per3, Per2/Per3 (double KD), and Per1/Per2/Per3 (triple KD) in MMH-D3 hepatocytes. All single knockdowns led to short periods in all three cell types, consistent with Synergy assays. Per1/Per2 double and Per1/Per2/Per3 triple knockdowns led to arrhythmicity. All other double composite knockdowns caused short period phenotype. A histogram of period length phenotypes is shown (bottom right panel). SD, 3 independent samples. NS, non-specific shRNA.

### DETAILED DESCRIPTION OF THE INVENTION

The invention features recombinant cells comprising detectable reporters that facilitate high temporal resolution quantitative luminescence recording and methods of using such cells to identify modulators of circadian period length and amplitude.

The invention is based, at least in part, on the discovery of new reporter cell lines, including NIH-3T3 (fibroblasts, commonly used clock model) 3T3-L1 (pre-adipocytes derived from 3T3 and can be differentiated into adipocytes for study), and MMH-D3 (hepatocytes when differentiated in culture). Lumicycle assays show that each model has high-amplitude and persistent rhythms that are amenable to high-throughput screening. These cell models facilitate clock gene characterization using RNAi and kinetic luminescence recording.

### Clock Biology

Much of what is known about the biochemistry and cell biology of the clock mechanism is based on two cellular models —NIH3T3 (mouse fibroblast) cells and U2OS (human osteosarcoma) cells. An implicit assumption in most circadian studies is that the clock works the same way in all cell and tissue types, and gene function determined in one cell type is generally considered to apply universally in all cells. This is not necessarily true. Several lines of evidence suggest that clock genes have tissue-specific functions. First of all, the SCN cell ensemble comprises a clock that is remarkably more robust than cultured fibroblasts and peripheral tissues lacking functional intercellular coupling. Second, when cultured in vitro or ex vivo, different tissues and cell types display different intrinsic period lengths and rhythm amplitudes. Furthermore, circadian mutants display different phenotypes in different tissues or cell types. For example, as reported herein below, Per1, Per2 and Per1 appear to have swapped their roles in different tissues.

As the cell is the simplest unit with circadian oscillations in mammals, cell-based models are the most efficient for its study. To address tissue specificity, several new cellular models of circadian clocks were developed: 3T3-L1 (pre-adipocytes derived from 3T3-cells, which can be differentiated into adipocytes.

To induce differentiation, 3T3-L1 preadipocytes are cultured in DMEM containing 10% FBS and antibiotics. Once cells reach confluence (day 0), differentiation is induced by supplying growth medium supplemented with 1 uM dexamethasone, 0.5 mM isobutylmethyxanthine (IBMX), and 1 ug/ml insulin. On day 2, the medium is replaced with DMEM supplemented with 10% FBS and 1 ug/ml insulin. The cells are subsequently re-fed every 48 hour with DMEM supplemented with 10% FBS. On day 7, cells are ready for experiments. Such methods are known in the art and described, for example, by Kallen and Lazar, Antidiabetic thiazolidinediones inhibit leptin (ob) gene expression in 3T3-L1 adipocytes. Proceedings of the National Academy of Sciences of the United States of America, 1996, 93(12):5793-6);

MMH-D3 hepatocytes are cultured in RPMI supplemented with 10% FBS, EGF, IGF-II and insulin, and antibiotics. Once cells reach confluence (day 0), differentiation is initiated by adding growth medium supplemented with 2% DMSO. The cells are subsequently re-fed every 48 hour. On day 9, cells were ready for experiments. Such methods are known in the art and described, for example, by Amicone et al, Transgenic expression in the liver of truncated Met blocks apoptosis and permits immortalization of hepatocytes. EMBO J, 1997, 16(3):495-503.

Each of these cells facilitate clock analysis because they display high-amplitude and persistent rhythms of reporter expression. Unlike tissue or animal models, these reporter cell lines are amenable to high-throughput screening. Establishing the tissue-specific function of clock genes has important implications. Such cells are useful for the identification of clock modifiers, and to identify which genes are potential core clock components regulating the SCN clock and animal behavior, and which are important for peripheral clock function and local physiology. Cells of the invention facilitate the characterization of the role that clock modulators play in altering distinct clock parameters, such as period length and amplitude.

### Screening Assays

The invention provides cellular compositions (e.g., fibroblasts, adipocytes, hepatocytes, and progenitors of these cell types) comprising a detectable reporter whose expression cycles with a circadian rhythm. In particular, as reported herein below, the invention provides cells comprising the Period gene promoter, Cry1 gene promoter, Cry1-Intron promoter, and Bmal1 promoters that are operably linked to luciferase.

Methods of the invention are useful for the high-throughput low-cost screening of candidate agents (e.g., inhibitory nucleic acids such as shRNAs, polypeptides, polynucleotides, small compounds) that modulate the expression (e.g., amplitude, period) of a detectable reporter in a cell of the invention. In one embodiment, an shRNA that modulates the circadian rhythm of a cell of the invention is identified as a clock modulator. The gene targeted by the identified shRNA is then characterized as a potential clock component. One skilled in the art appreciates that the effects of a candidate agent on a cell is typically compared to a corresponding control cell not contacted with the candidate agent. Thus, the screening methods include comparing the rhythmicity of expression of a detectable reporter (e.g., amplitude, period) in a cell contacted by a candidate agent to the expression of an untreated control cell.

In another embodiment, cells of the invention are used to determine potential adverse effects of pharmacological drugs on circadian clock function. The drugs may be proprietary, or commercially available and are being administered to patients of various diseases such as diabetes, obesity and cardiovascular diseases. Those that have effects on clock function in our cell type-specific models would provide entry points for testing drug effects on human clock function, such as changes in sleep patterns in patients.

In other embodiments, cells of the invention are used to determine the optimal time for drug administration to a subject. For example, a cell of the invention is contacted with an agent at various time points over the course of the day, and the agent's effect on cell physiology is assayed to determine whether the agent's efficacy or probability of causing adverse side effects alters as a function of the time of administration. The cellular physiology of potential interest in the context of fibroblasts, adipocytes and hepatocytes ranges from RNA and protein production, membrane transport, autophagy and cell division, to cell signaling, cell death, and metabolism. In particular, for example, hepatocytes can be used to study effects of differential temporal application of antidiabetic drugs such as Metformin and TZD, on cellular physiology such as insulin sensitivity, glycogen synthesis and gluconeogenesis, as well as on detoxification and metabolism of xenobiotics.

The effects of agents on a cell's circadian rhythm can be assayed by detecting the expression or activity of a Period, Cry1, Cry2, Cry3, or Bmal1 polypeptide or polynucleotide. Polypeptide or polynucleotide expression can be detected by procedures well known in the art, such as Western blotting, flow cytometry, immunocytochemistry, binding to magnetic and/or antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents are added at varying concentrations to the culture medium containing a cell of the invention. An agent that modulates the expression of detectable reporter expressed in the cell is considered useful in the invention; such an agent may be used, for example, as a clock modulator. An agent identified according to a method of the invention is locally or systemically delivered to modulate the circadian rhythm of a subject.

If one embodiment, the effect of a candidate agent may be measured at the level of polypeptide production using the same general approach and standard immunological techniques, such as Western blotting or immunoprecipitation with an antibody specific for Period, Cry1, 2, or 3, or Bmal1. For example, immunoassays may be used to detect or monitor the expression of protein of interest in a cell of the invention.

Alternatively, or in addition, candidate agents are identified by first assaying those that modulate the reporter expression of a cell of the invention and subsequently testing their effect on cells of the SCN, or on whole animals, which would have implications in human diseases. In one embodiment, a clock modulator polypeptide is assayed for its ability to interact with Clock polypeptides, for example, using Gal4 two-hybrid screen as described herein. Such interactions can also be readily assayed using any number of standard binding techniques and functional assays (e.g., those described in Ausubel et al., supra).

### Inhibitory Nucleic Acids

Inhibitory nucleic acid molecules are those oligonucleotides that inhibit the expression or activity of a polypeptide. Such oligonucleotides include single and double stranded nucleic acid molecules (e.g., DNA, RNA, and analogs thereof) that bind a nucleic acid molecule of interest (e.g., antisense molecules, siRNA, shRNA), as well as nucleic acid molecules that bind directly to the polypeptide to modulate its biological activity (e.g., aptamers). siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as clock modulators.

The inhibitory nucleic acid molecules of the present invention may be employed as double-stranded RNAs for RNA interference (RNAi)-mediated knock-down of expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 800 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and such vectors allow for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Test Compounds and Extracts

In general, clock modulators are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include those known as therapeutics for the treatment of pathogen infections. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have clock modulating activity, further fractionation of the positive lead extract is necessary to isolate molecular constituents responsible for the observed effect. Thus, the goal of the extraction, fractionation, and purification process is the careful characterization and identification of a chemical entity within the crude extract that modulates period or amplitude. Methods of fractionation and purification of such heterogenous extracts are known in the art. If desired, compounds shown to be useful as therapeutics are chemically modified according to methods known in the art.

### Therapeutic Methods

Agents identified as clock modulators are useful, for example, in improving the body's circadian rhythms in physiology and behavior through adjustment of clock properties, including resetting/synchronization of the clocks with the environment and throughout the body, and changes in period length and amplitude of various circadian rhythms of our body, or otherwise ameliorating symptoms associated with jet lag, seasonal affective disorder, shift work- and sleep-related disorders, and metabolic syndromes associated with clock disorders.

In one therapeutic approach, an agent identified as described herein is administered to a tissue comprising cells that cycle with a circadian rhythm (e.g., suprachiasmatic nucleus, liver, fat cells) or is administered systemically. The dosage of the administered agent depends on a number of factors, including the size and health of the individual patient. For any particular subject, the specific dosage regimes should be adjusted over time according to the individual need and the professional judgment of the person administering or supervising the administration of the compositions.

### Delivery of Polynucleotides

Naked polynucleotides, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### A New Cellular Model of Circadian Clock Function

To inform the molecular, cellular, and physiological mechanisms of the clock, new cellular models are required. Complex genetic perturbations, for example, dose-dependent or pairwise, are difficult or impossible to identify in animal models, and are too time-intensive and expensive to support whole genome screening, for example, using RNA interference approaches to identify synthetic small interfering RNAs (siRNAs) modulators of circadian rhythms. The present invention provides cellular models that are easily cultured and amenable to transfection and/or infection and quantitative luminescence recording including real-time imaging when necessary, and, most importantly capable of generating robust circadian rhythms in vitro.

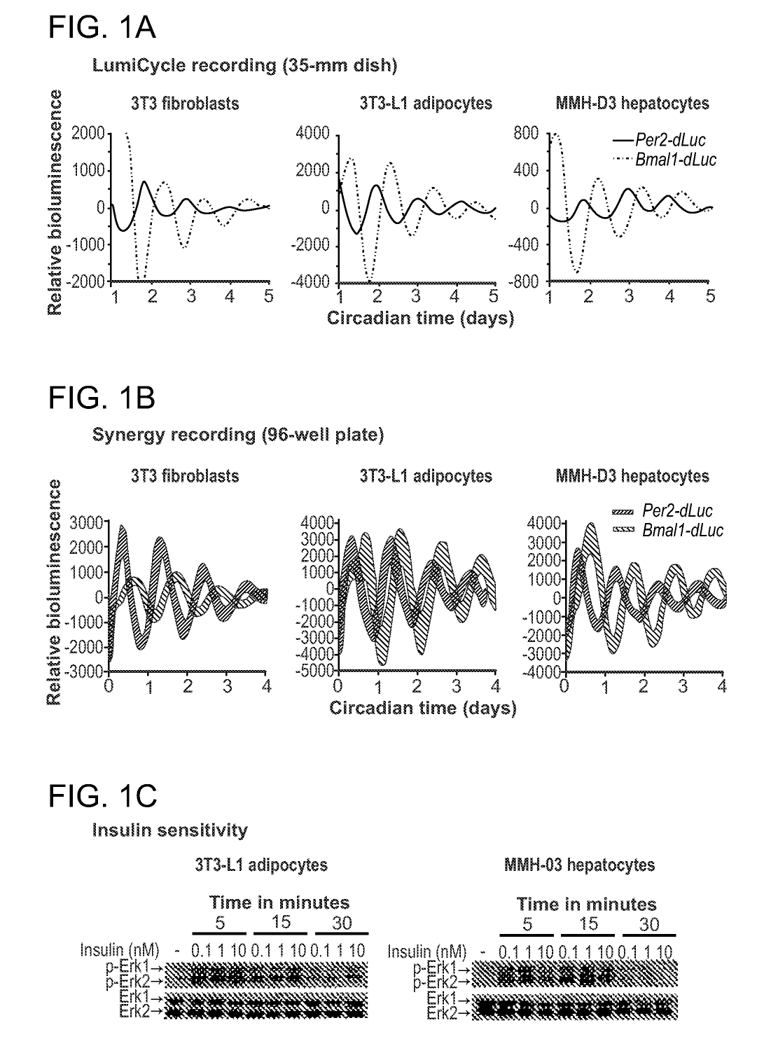

To develop new models, a battery of luciferase-based reporters that can be introduced into cells via transient transfection or lentiviral transduction was engineered. To explore cell type specificity of clock gene function, new reporter cell lines comprising lentiviral luciferase reporter driven either by the Per2 or Bmal1 promoters were developed. Cell comprising these vectors include NIH-3T3 (fibroblasts, commonly used clock model, 3T3-L1 (pre-adipocytes derived from 3T3 that can be differentiated into adipocytes for study, and MMH-D3 (hepatocytes when differentiated in culture). Lumicycle assays show that each model has high-amplitude and persistent rhythms (FIG. 1A). Further, single cell cloning from these reporter cells was performed, and isolated clonal cells showed similar rhythm amplitude and period length as cell mass but displayed much higher levels of bioluminescence expression, necessary for high-throughput assays. The rhythm assays using these clonal cell lines were adapted onto 96-well plates using a Synergy luminometer recording device. Unlike tissue or animal models, these clonal reporter cell lines are amenable to high-throughput screening (i.e., genetic perturbation and rhythm assay in 96- or 384-well format) (FIG. 1B). Importantly, these differentiated cells display insulin sensitivity (FIG. 1C), indicative of the basic physiological properties of these cells.

In sum, these adipocyte and hepatocyte models provide new tools to ascertain the effect of genetic or environmental perturbations on clock function.

### Example 2

### Development of Lentiviral shRNA for Gene Knockdown

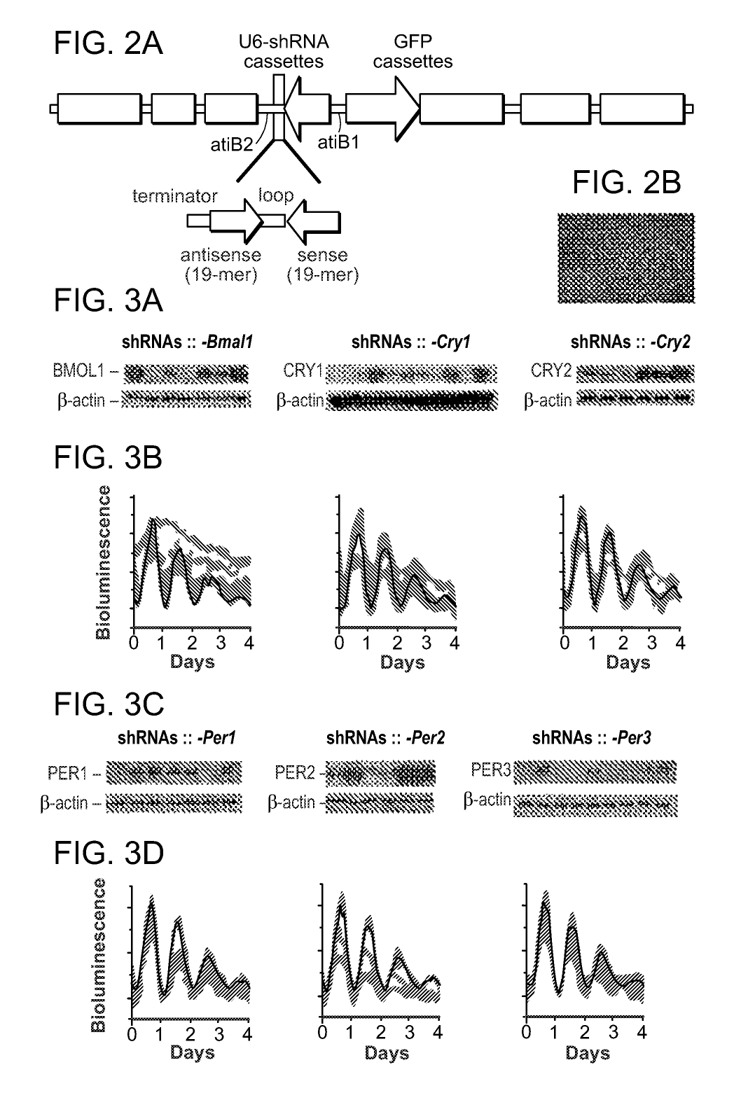

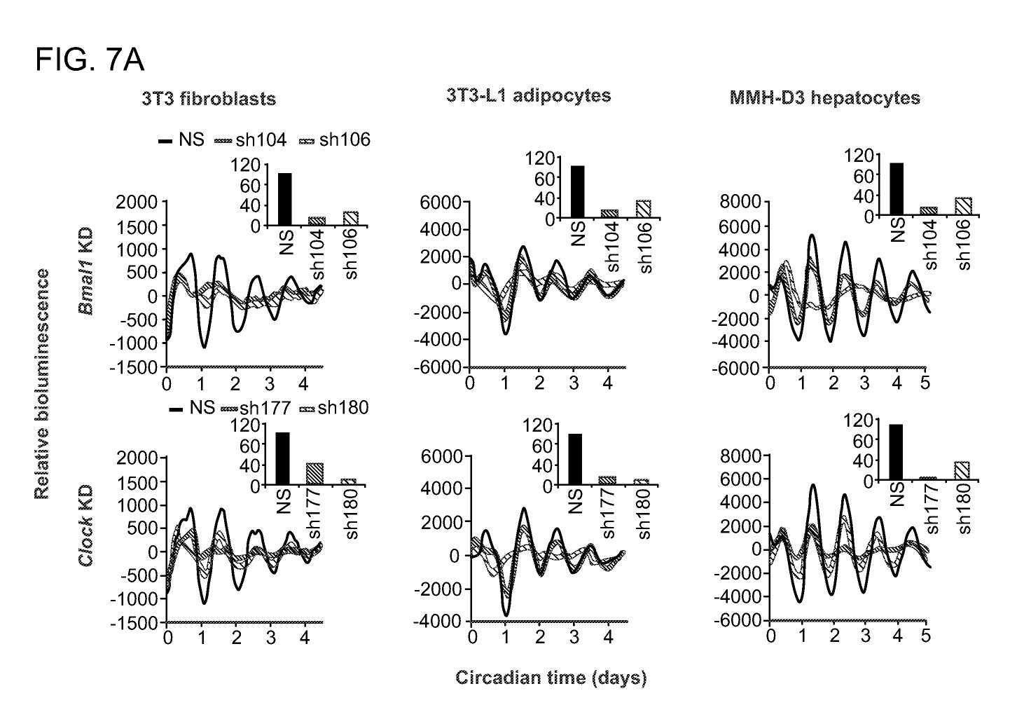

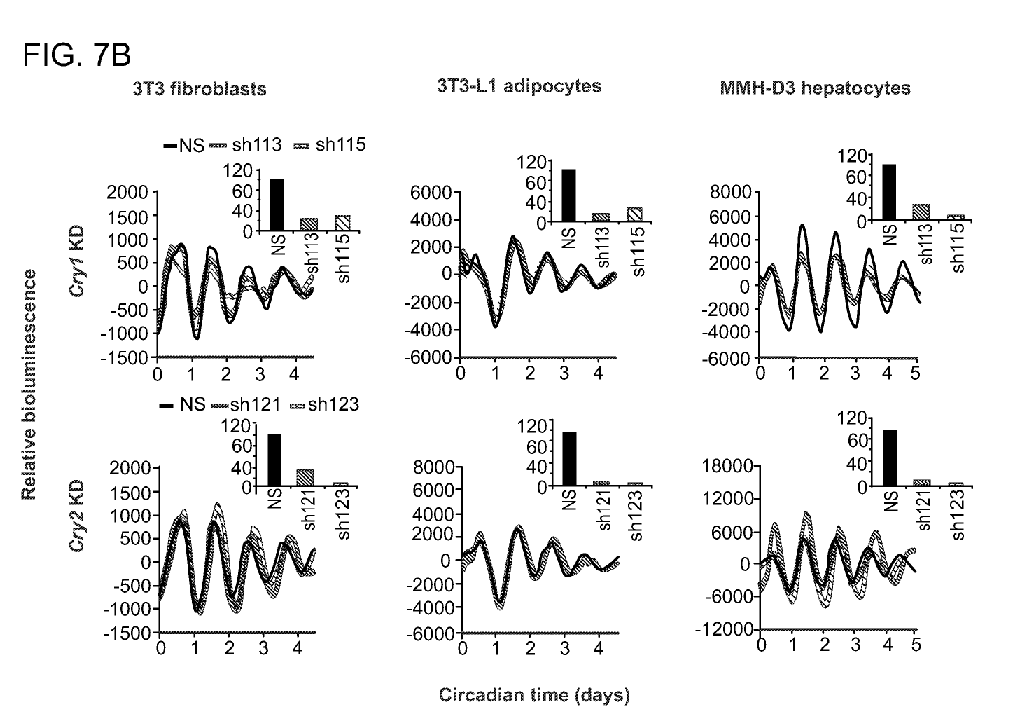

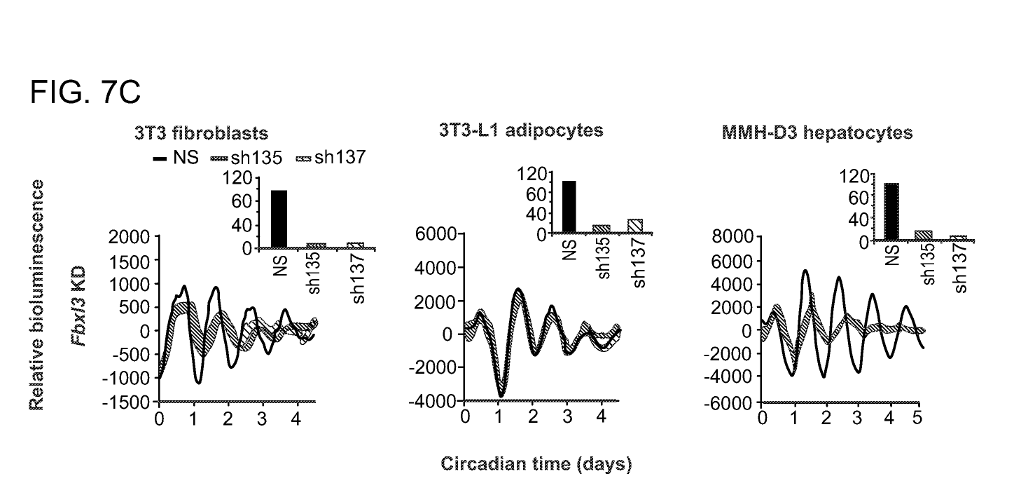

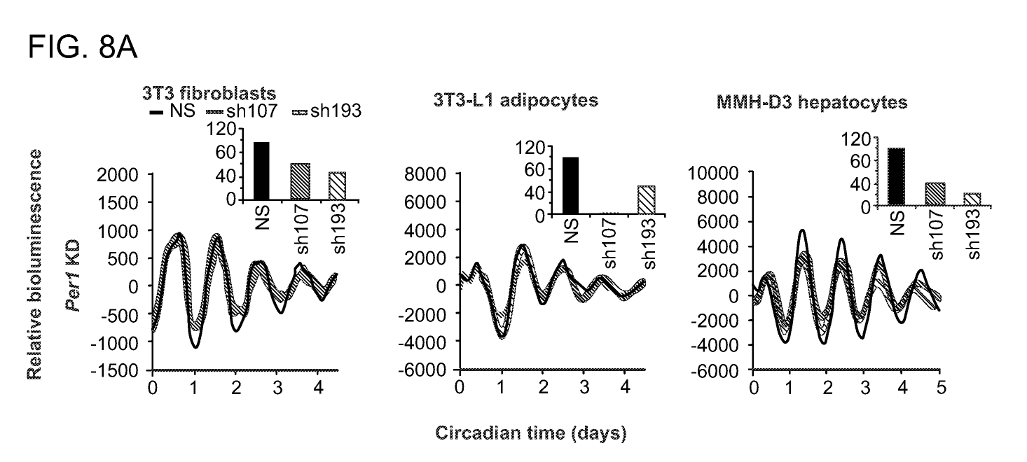

For genetic perturbation, a pipeline was developed to produce high-quality, validated lentiviral shRNA vectors to knock down murine clock genes. shRNAs were selected to facilitate both cell based assays and intact tissue slice preparations. In brief, 5 shRNA constructs against genes of interest were designed using an optimized shRNA design algorithm that selects for optimal target sequence for knockdown and against homologous sequences to minimize off-target effects. Oligos were synthesized and then cloned into pGWL-si2/U6 in which shRNA expression is under the control of the mouse U6 promoter. Subsequently, the U6-shRNA expression cassette was cloned into the lentiviral pLL3.7 Gateway vector (modified from pLL3.7) (FIG. 2A). Virus was prepared using standard methods and the efficacy of infection was estimated by observing co-expressed GFP from a CMV promoter (usually most cells are GFP positive after infection) (FIG. 2B). A panel of siRNAs was generated against all known clock factors. For each clock factor, at least two of the five candidate shRNAs were effective in knockdown (FIG. 2C)(FIGS. 7-9). These results demonstrate the feasibility of generating lentiviral shRNAs against virtually any clock gene of interest and validating the efficacy of these shRNA as clock modulators.

### Example 3

### Cell Type-Specific Function of Per1, Per2 and Per3

This approach was used to design shRNAs against Bmal1, Clock, Cry1, Cry2, and Fbxl3 (FIG. 7). Co-transfection of these shRNAs showed efficient knockdown of the proteins as determined by Western blot (FIG. 3A) and by quantitative PCR (FIG. 7). Knockdown of these genes resulted in expected phenotypes in all cell lines, consistent with previous studies. For example, knockdown of Bmal1 results in low-amplitude rhythms or arrhythmicity; and whereas Cry2 knockdown lengthens periods, Cry1 knockdown leads to short periods and/or rapid loss of rhythmicity (FIG. 3B).

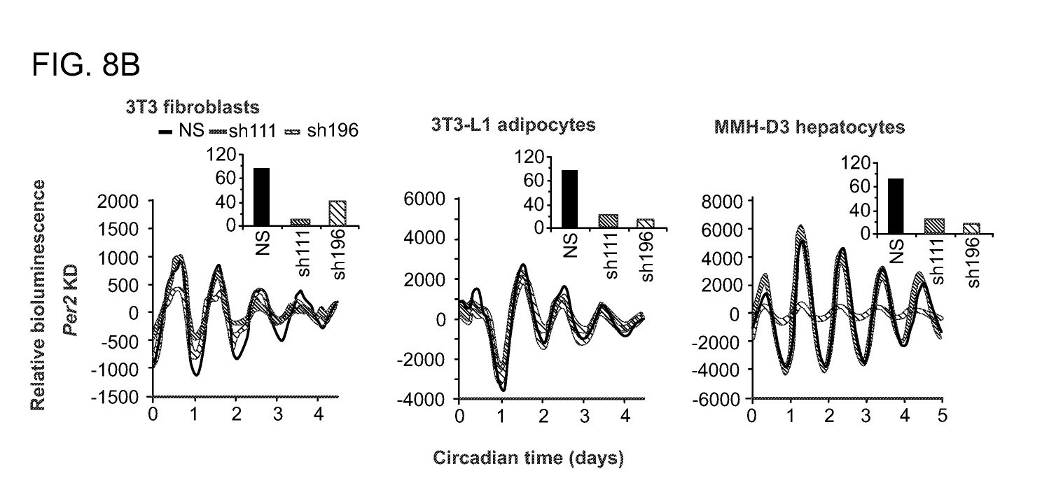

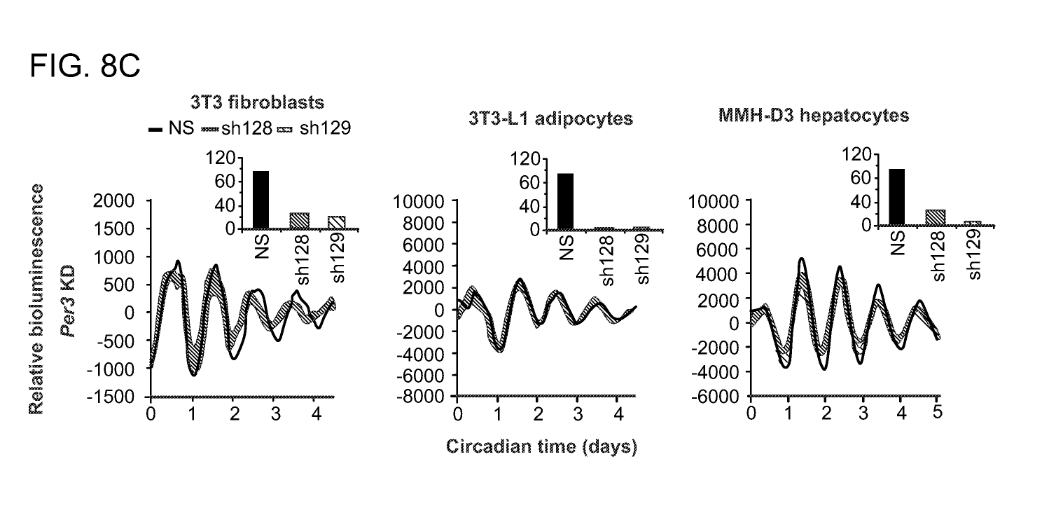

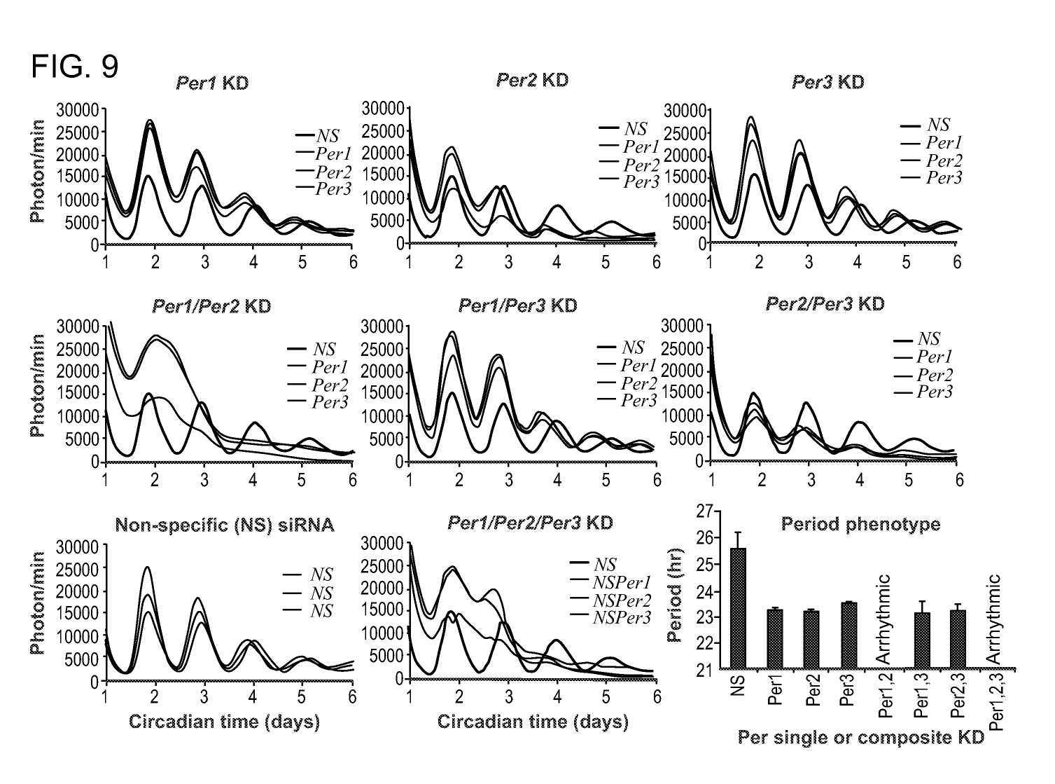

Similarly, shRNA constructs against Per1, Per2, and Per3 also effectively down-regulated gene and protein expression (FIG. 3C)(FIG. 8). Surprisingly, unexpected knockdown phenotypes were observed (FIG. 3D) (FIG. 8). First, unlike in other cell types or tissues, Per1 disruption had only mild effects in 3T3 and 3T3-L1 cells and short period lengths in MMH-D3 cells. Surprisingly, while Per3 deletion had only a subtle effect on the SCN clock and isn't considered part of the core clock, it produced strong phenotypes in each of the three cellular models described herein. The circadian phenotypes obtained from these knockdown studies and reported knockout data are summarized in Table 1 (below). The knockdown phenotypes were further confirmed in LumiCycle assays (FIG. 9). Importantly, double and triple knockdowns of Per1, Per2 and Per3 genes revealed their novel, relative contributions to the clock function in this hepatocyte cell type (FIG. 9).

TABLE 1
Summary of Per phenotypes.

| | | | | | | MEF/MAF | NIH-3T3 | 3T3-L1 | MMH-D3 |
|---|---|---|---|---|---|---|---|---|---|
| | SCN | Liver | Lung | Pituitary | U2OS | Fibroblasts | Fibroblasts | Adipocytes | Hepatocytes |
| Per1−/− | wt | AR/RD | AR/RD | AR/RD | AR/RD | AR/RD | wt | wt | short/RD |
| Per2−/− | short | ND | wt | wt | long/RD | AR/RD | short/RD | wt | wt/RD |
| Per3−/− | wt | short | short | short | short | short | shod | short | short |

1. Abbreviation: wt, wild type; AR, arrhythmic; RD, rapid damping or low amplitude; ND, not determined.
2. Cell lines: U2OS, human osteosarcoma; MEF, mouse embryonic fibroblasts; MAF, mouse adult tail fibroblasts.
3. Period changes that are <2 standard deviations from the mean are considered wt phenotype.
4. Results are from various reports (Liu A C, Welsh D K, Ko C H, Tran HG, Zhang E E, Priest A A, Buhr E D, Singer O, Meeker K, Verma I M, et al. Intercellular coupling confers robustness against mutations in the SCN circadian clock network. Cell 2007 May 4; 129(3): 605-16; Baggs J E, Price T S, DiTacchio L, Panda S, FitzGerald G A, Hogenesch J B. Network features of the mammalian circadian clock. PLoS Biol 2009 Mar 10; 7(3): e52; Pendergast J S, Friday R C, Yamazaki S. Endogenous rhythms in Period1 mutant suprachiasmatic nuclei in vitro do not represent circadian behavior. J Neurosci. 2009 Nov 18; 29(46): 14681-6; Pendergast J S, Friday R C, Yamazaki S. Distinct functions of Period2 and Period3 in the mouse circadian system revealed by in vitro analysis. PLoS ONE. 2010; 5(1): e8552; Pendergast J S, Niswender K D, Yamazaki S. Tissue-specific function of Period3 in circadian rhythmicity. PLoS One. 2012; 7(1): e30254. and from our preliminary study.

As evident from the summary, the Per genes appear to swap roles depending on cell types, with Per1 and Per2 functioning prominently in the SCN, while Per3 functions primarily in peripheral oscillators.

### Example 4

### Protein Interactions with Known Clock Components

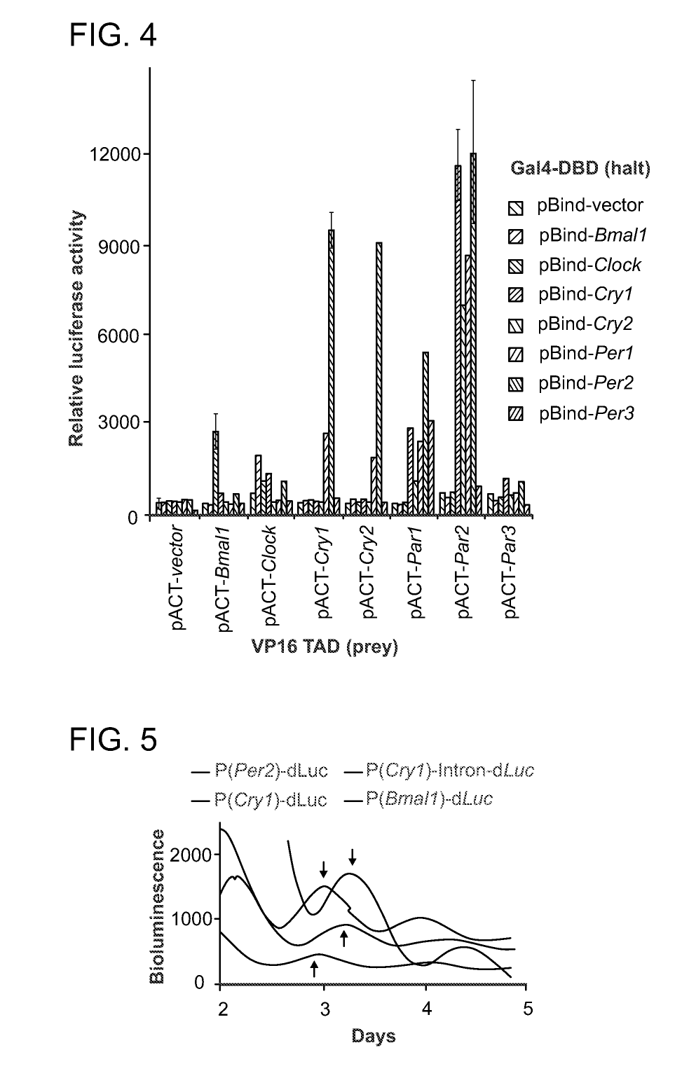

A hallmark of circadian clocks is the time-dependent formation of clock protein complexes. To determine if identified clock modulators physically interact with known clock components, a mammalian two-hybrid assay is used. To this end, a collection of fusion proteins for each core clock component has been generated. In one set, each protein is tagged with the DNA-binding domain of the yeast Gal4 protein. In the second set, each protein is fused with the mammalian coactivator, VP16. When co-transfected into mammalian cells with a reporter containing Gal4 binding site driving luciferase expression, if the two proteins interact (e.g., Bmal1 and Clock), they will bring into close proximity the DNA-binding domain of Gal4 and the trans-activation domain of VP16 and drive robust transcription. If not, no transcription will be activated. This assay is extraordinarily sensitive and takes advantage of the native mammalian cellular environment. This assay to validate interactions among known clock components (FIG. 4). In most cases, the interactions are reciprocal (e.g., Cry1 and Cry2 with Per1 and Per2).

This system can also be used to test novel clock modifiers for interaction with known clock factors. To confirm endogenous interactions with clock proteins, Co-IP and Western blot analysis is performed.

### Example 5

### Reporters Showing Four Circadian Phases of Gene Expression

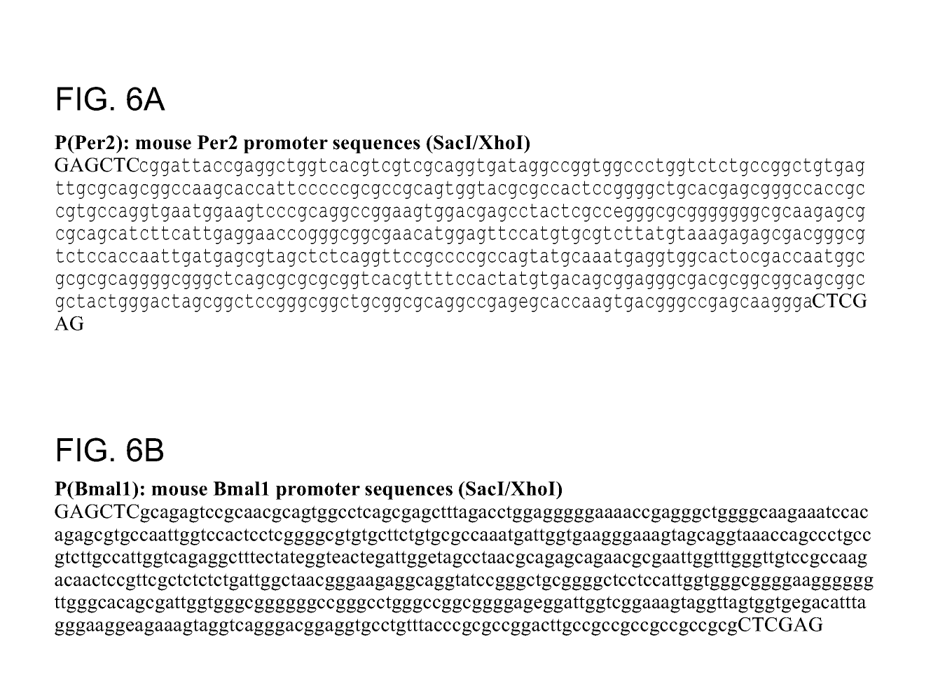

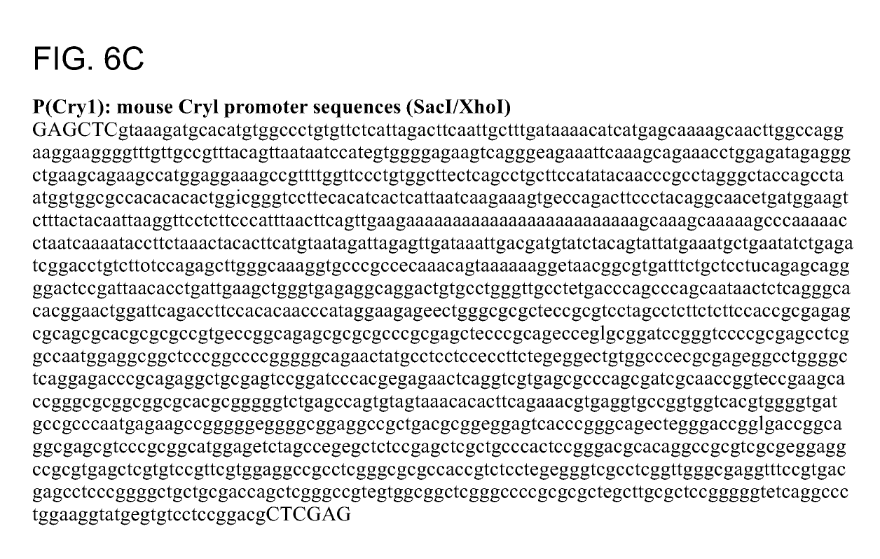

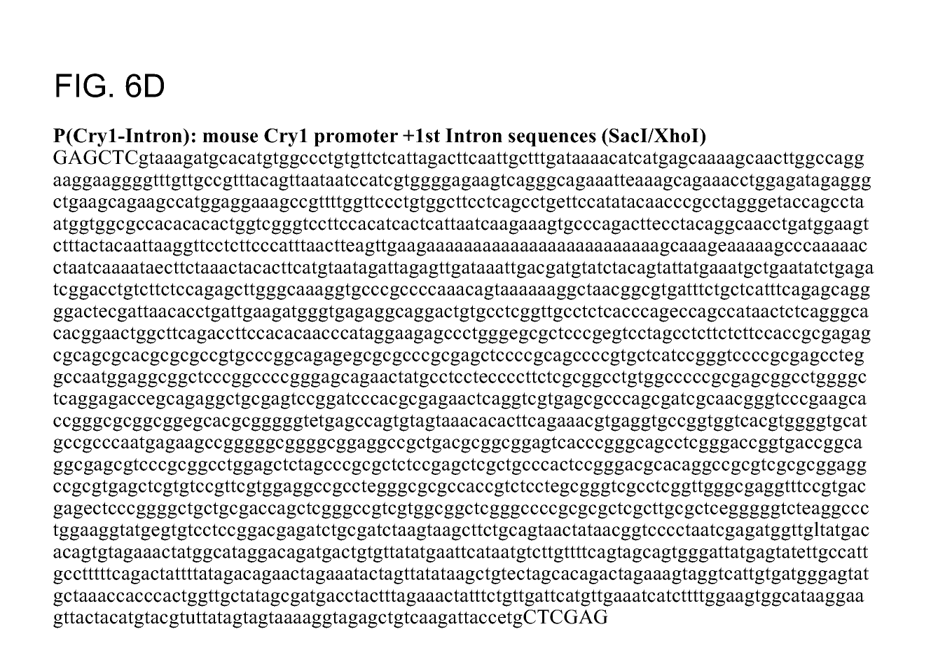

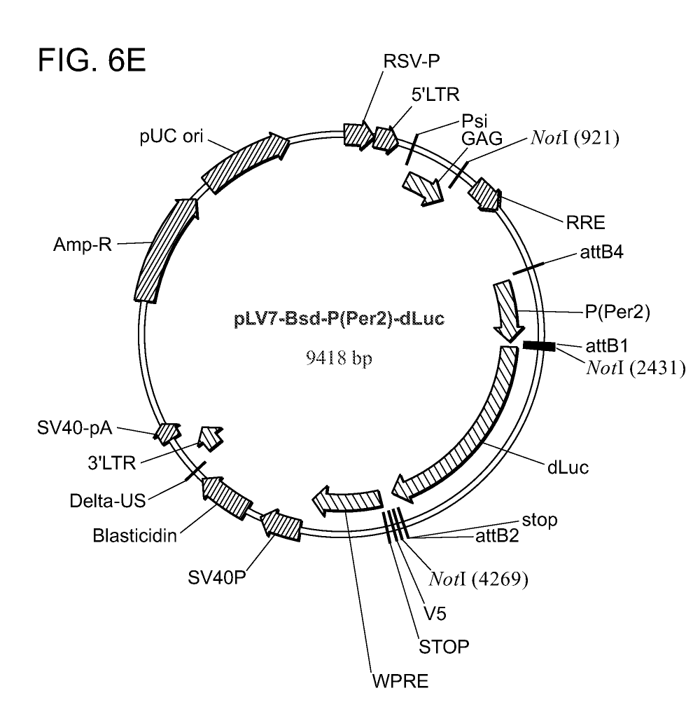

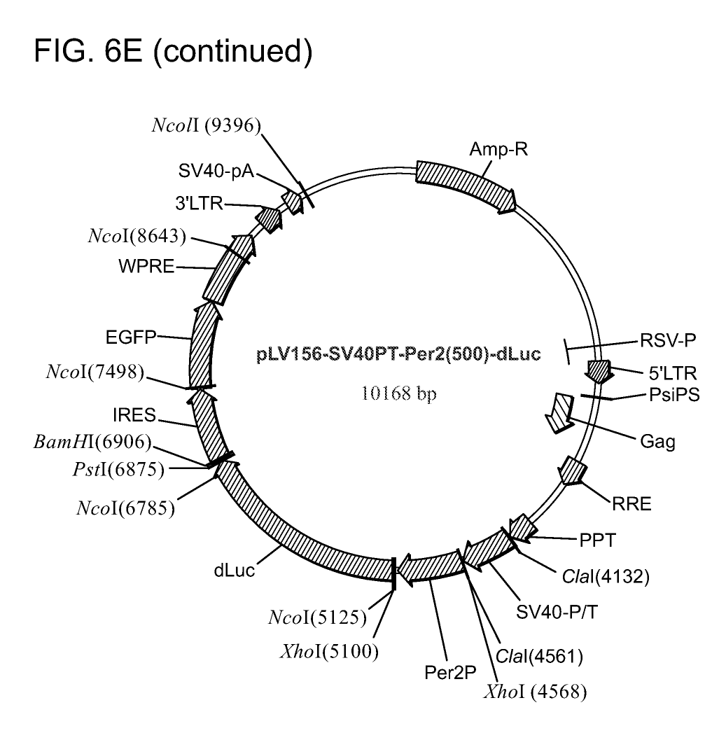

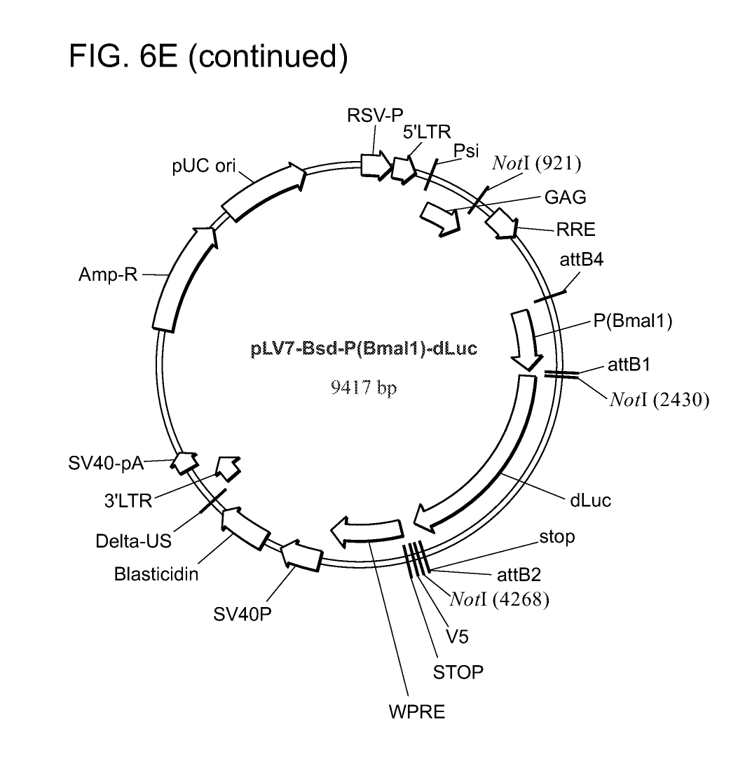

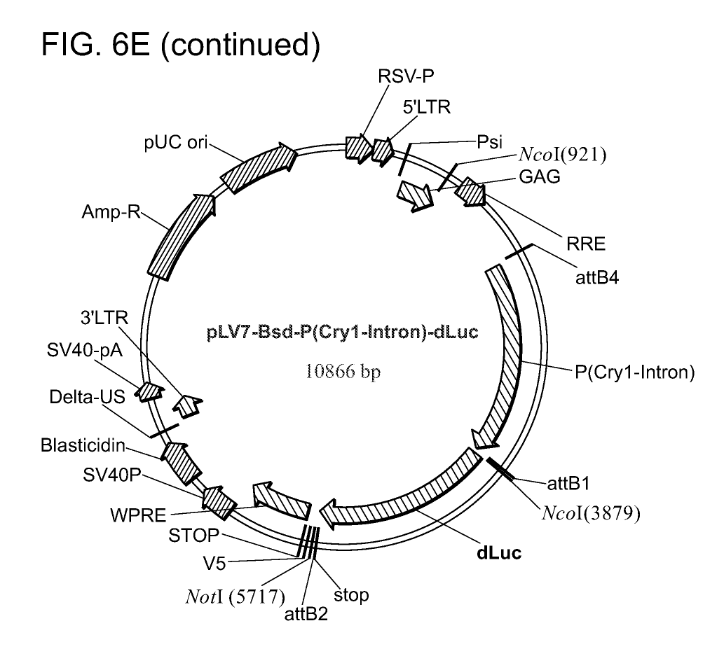

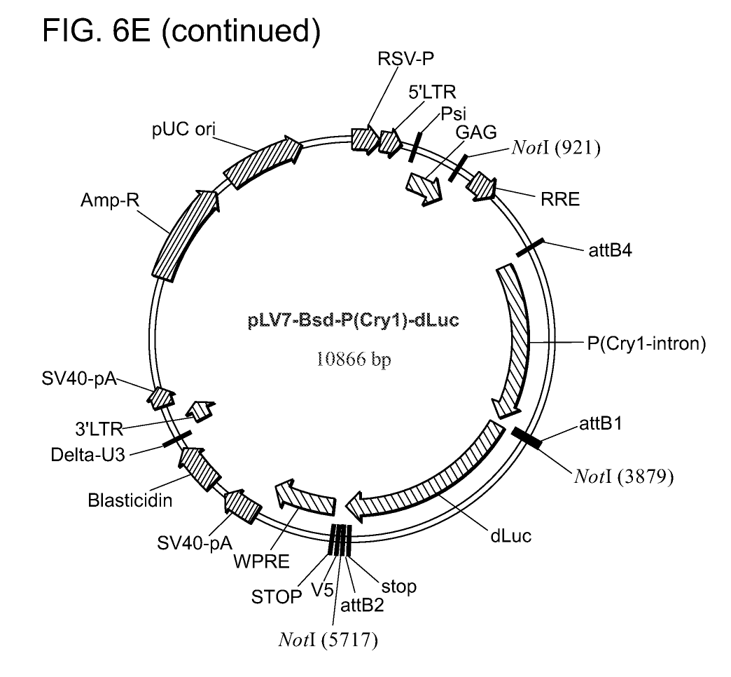

At least four circadian phases of gene expression have been recapitulated in cultured mammalian cells: P(Per2)-dLuc, P(Cry1)-dLuc, P(Cry1)-Intron-dLuc, and P(Bmal1)-dLuc (FIG. 5). Each of these four reporters has been introduced into each of the three cell lines (FIGS. 6A-6E). To study the role of each novel gene in each cell type, a validated shRNA against a candidate will be introduced into reporter cells, followed by a rhythm assay. From rhythm data, clock phenotypes will be identified, and the amplitude and phase of the reporters in the presence or absence of the specific shRNA will provide mechanistic insights into the clock.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A recombinant cell comprising an expression vector, wherein the expression vector comprises a promoter selected from the group consisting of Period2 (Per2), Cry1, Cry1-Intron, and Bmal1, wherein the promoter is operationally linked to a detectable reporter that is expressed at high-amplitude and with a persistent rhythm.

2. A recombinant adipocyte or hepatocyte cell or progenitor thereof comprising an expression vector, wherein the expression vector comprises Period2 (Per2), Cry1, Cry1-Intron, and Bmal1 promoter operationally linked to a detectable reporter.

3. The recombinant cell of claim 1 or 2, wherein the cell is a 3T3-L1 pre-adipocyte or a MMH-D3 pre-hepatocyte.

4. The recombinant cell of claim 1 or 2, wherein the expression vector is a lentiviral vector.

5. The recombinant cell of claim 1 or 2, wherein the detectable reporter is a luciferase reporter.

6. The recombinant cell of claim 1 or 2, wherein the reporter expression varies at least about two to four fold in trough to peak levels.

7. The recombinant cell of claim 1 or 2, wherein the reporter expression varies at least about three fold in trough to peak levels.

8. A method of identifying a circadian cycle modulator, the method comprising contacting the cell of any of claims 1-7 with an agent, and assaying reporter expression in the contacted cell relative to a corresponding control cell.

9. The method of claim 8, wherein the agent is a small compound, inhibitory nucleic acid, or polypeptide.

10. A method of identifying a circadian cycle modulator, the method comprising contacting the cell of any of claims 1-7 with an shRNA against a gene of interest, and analyzing a circadian rhythm of the cell relative to a reference, thereby identifying a circadian cycle modulator.

11. The method of claim 9 or 10, wherein the circadian rhythm of the cell is analyzed by detecting the amplitude, period length and phase of reporter expression.

12. The method of claim 9 or 10, wherein the reference is the circadian rhythm of an untreated control cell.

13. The method of claim 9 or 10, wherein the circadian rhythm is analyzed using luminescence recording, and/or real-time imaging.

14. The method of claim 9 or 10, wherein the circadian cycle modulator is an inhibitory nucleic acid molecule, small compound, or polypeptide.

15. The method of claim 9 or 10, wherein the inhibitory nucleic acid molecule is an shRNA.
